(19) United States
(12) Patent Application Publication
(10) Pub. No.: US 2025/0281683 A1
(43) Pub. Date: Sep. 11, 2025

(54) OXYGENATOR WITH INTEGRATED PRESSURE SENSOR

(71) Applicant: CHINABRIDGE (SHENZHEN) MEDICAL TECHNOLOGY CO., LTD., Shenzhen (CN)

(72) Inventor: Andrea Zaniboni, Mirandola (IT)

(21) Appl. No.: 18/601,894

(22) Filed: Mar. 11, 2024

(51) Int. Cl. A61M 1/16; A61M 1/14

(52) U.S. Cl. CPC A61M 1/1698; A61M 1/153; A61M 1/1625; A61M 1/1629; A61M 2205/18; A61M 2205/3331; A61M 2205/3592; A61M 2205/583; A61M 2230/30

(57) ABSTRACT

An oxygenation device for use in connection with extracorporeal blood circulation, the device including a stacked oxygenator housing defining a blood inlet, a blood outlet and a blood flow path between the blood inlet and the blood outlet. The device further includes a plurality of stacked hollow fibers disposed inside the housing and along the blood flow path, the hollow fibers fluidly coupled to a gas inlet port and a gas outlet port. The blood inlet port has a blood inlet lumen fluidly coupled to the blood inlet and includes an inlet housing in fluid communication with the blood inlet lumen, the inlet housing adapted to house an inlet pressure sensor. The blood outlet port has a blood outlet lumen fluidly coupled to the blood outlet and includes an outlet housing in fluid communication with the blood outlet lumen, the outlet housing adapted to house an outlet pressure sensor.

### TECHNICAL FIELD

This disclosure relates to the field of devices for extracorporeal circulation of blood. More specifically, the disclosure relates to some important components of the extracorporeal circuit, such as the sensors, in particular pressure sensors used to measure the blood pressures at various points of the circuit.

### BACKGROUND

Blood extracorporeal circuits may include dual portions of an oxygenator and heat exchanger to exchange oxygen and carbon dioxide between blood and a gas mixture and exchange heat between blood and a heating or cooling fluid through the walls of semipermeable hollow fiber membranes. Such circuits also include other elements like a pump for providing a certain blood flow through the circuit and sensors measuring quantities, among others, like blood pressures, flow rate, temperatures, oxygen saturations and oxygen and carbon dioxide partial pressures. Blood contacts the outside surfaces of the hollow fibers, while the gas mixture and the heating/cooling fluid (e.g., a water solution) are circulated inside the hollow fiber lumens. In the devices using this technology, the hollow fibers may be organized in different ways. They may be in a single or multifilament form which is woven (to form a so-called “wound” oxygenator type) around a core or may be structured in mats wound around a core or stacked in parallel mat layers on top of one another without a core (to form a so-called “stacked” oxygenator type). Various examples of this technology are well known in the technical field. As far as the blood pump it may be of roller, or centrifugal types. In the latter case, the pump rotor may be levitated and kept floating by means of a magnetic field, which results in low friction operation and hence reduced hemolysis rate, particularly necessary in long term perfusion procedures like in ECMO (ExtraCorporeal Membrane Xxygenation), lasting several days, if not a few weeks, of continuous extracorporeal heart/lung support of the patient. In ECMO, very often the extracorporeal circuit needs to be moved with the patient from one hospital department to another and thus it's important that it be as much simplified, compact and lightweight as possible.

Often measuring sensors are used to keep the process under control, which results in adding to the complexity of the system, particularly when the sensors are reusable and cannot come into direct contact with blood. In the case of reusable pressure sensors, it is necessary to interpose blood catchers, mounted along specific tubing lines derived from the main flow circuit and placed at the pressure measuring points to create small air separation volumes between blood and sensors to prevent blood from touching the sensors. Such a complication is overcome with disposable pressure sensors which are discarded with the entire extracorporeal circuit at the end of the perfusion procedure and are included in expressly made connectors inserted along the main tubing lines to and from the patient, each of them with the sensor area in direct contact with blood by means of a small hole drilled through the tube wall. With them, the complexity is decreased as there are no more derived measuring lines and blood catchers are no more needed. In any case, the pressure sensor connectors are somewhat bulky elements added to the system, requiring a certain space along the tubing in correspondence to the appropriate measuring locations. This may pose circuit layout problems when such a space is not sufficient and may oblige the operator to dispose the sensors away from the oxygenator blood ports, which would result in measuring at locations not ideal for optimally controlling performance of the extracorporeal circuit. Also, electrical connections between the sensors and the remote monitoring unit may present a complexity problem.

### SUMMARY

Embodiments of the present invention include an oxygenation hollow fiber membrane organized in stacked hollow fiber mat layers with a structure expressly conceived to provide higher-gas exchange efficiency and better oxygenation effect with pressure sensors integrated into the blood inlet and outlet ports.

The invention here proposed provides increased simplification and compactness to the extracorporeal circuit, obtained by integrating the disposable pressure sensors into the inlet and outlet ports of a stacked oxygenator device. In this way, oxygenator and pressure sensors become one integrated assembly, thus reducing the number of circuit assembling operations and increasing user friendliness during set up and initial prime of the system. In a stacked oxygenator, the pressures are measured in positions that are relatively close, respectively, to the blood inlet and the blood outlet of the gas transfer compartment of the oxygenator. By monitoring these blood inlet and blood outlet pressures, it is possible to receive an early indication of oxygenator malfunction. For instance, a blood pressure drop (given by DeltaP=Pin−Pout, where Pin is the pressure measured at the oxygenator blood inlet port and Pout is the pressure measured at the oxygenator blood outlet port) increase may be indicative of coagulation starting inside the oxygenator and thus may trigger a series of corrective actions from the operator.

In other words, such integration provides the oxygenator “true” inlet/outlet pressure and pressure drop values that would not be otherwise obtainable if the two sensors, as is usually the case with would oxygenators, cannot be integrated close to the inlet and outlet blood ports, and thus were connected along the tubing, each of them placed at a certain distance apart from the oxygenator. In such situations, the measured values would in fact be inclusive also of the pressure drops associated with the paths between the sensors and the oxygenator ports. Another advantage of this integration is that it allows rationalized and simplified electrical connections of the pressure sensors to the remote control and monitoring unit which displays and monitors the pressure values. The connection may be realized in various ways, e.g., by two cables (each connected to the electrical socket of each integrated sensor), or by only one cable connected to a single socket wired to both integrated sensors and conveniently placed on the oxygenator housing, or without cables by exploiting a wireless (e.g., Wi-Fi or Bluetooth) connection between oxygenator and remote control and monitoring unit. In the case of connection to the remote control and monitoring unit by one or two cables, the pressure sensor socket(s) may be oriented in any directions with respect to the oxygenator. In the case of wireless connection, a battery-operated transmitter conveniently placed on the oxygenator housing, and wired to both integrated sensors, communicates the measured pressure values by telemetry to the remote control and monitoring unit, thus further increasing the compactness of the system.

Example 1 is an oxygenation device for use in connection with extracorporeal blood circulation, the device including an oxygenator housing including a blood inlet end cap defining a blood inlet opening, a blood outlet end cap defining a blood outlet and a blood flow path between the blood inlet and the blood outlet; a plurality of stacked, mat layers of hollow fibers disposed inside the housing and along the blood flow path, the hollow fibers fluidly coupled to a gas inlet port and a gas outlet port; a blood inlet port having a blood inlet lumen defining an inlet sensing hole and fluidly coupled to the blood inlet opening of the blood inlet end cap, the blood inlet port including an inlet pressure sensor housing in fluid communication with the blood inlet lumen through the inlet sensing hole, the inlet pressure sensor housing adapted to house an inlet pressure sensor, wherein the inlet sensing hole is disposed less than 3 cm from the blood inlet opening on the blood inlet end cap; and a blood outlet port having a blood outlet lumen fluidly defining an outlet sensing hole and fluidly coupled to the blood outlet opening of the blood outlet end cap, the blood outlet port including an outlet pressure sensor housing in fluid communication with the blood outlet lumen through the outlet sensing hole, the outlet pressure sensor housing adapted to house an outlet pressure sensor; wherein during operation of the oxygenation device, each of the inlet pressure sensor and the outlet pressure sensor are configured to measure an inlet blood pressure and an outlet blood pressure, respectively.

Example 2 is the device of Example 1, wherein the oxygenator housing defines an oxygenator module and a heat exchanger module, and further wherein the plurality of hollow fibers includes oxygenator fibers associated with the oxygenator module and heat exchanger fibers associated with the heat exchanger module.

Example 3 is the device of Example 2, wherein the housing further comprises a separation grid or a collection grid positioned between the heat exchanger module and the oxygenator module.

Example 4 is the device of Example 1 further comprising a remote monitoring unit communicatively coupled to each of the inlet and outlet pressure sensors.

Example 5 is the device of Example 4 further comprising communication circuitry coupled to each of the inlet and outlet pressure sensors and adapted to communicate with the remote monitoring unit.

Example 6 is the device of Example 5, wherein the communication circuitry is adapted to communicate with the remote monitoring unit by wireless telemetry.

Example 7 is the device of Example 1 further comprising electrical terminals mechanically connected to each of the inlet and outlet ports and adapted for coupling at least one of the inlet and outlet pressure sensors with an electrical connector.

Example 8 is the device of Example 7, wherein the electrical connector is configured to communicatively couple both of the pressure sensors with a remote monitoring unit.

Example 9 is the device of Example 7 further comprising a second electrical connector and wherein the first electrical connector is adapted to couple the inlet pressure sensor to the remote monitoring unit and the second electrical connector is adapted to couple the outlet pressure sensor to the remote monitoring unit.

Example 10 is the device of Example 1, wherein the housing is configured such that a gas mixture may enter through the gas inlet port, pass through the plurality of hollow fibers and exit through the gas outlet port.

Example 11 is the device of Example 1, wherein the housing further defines a fluid inlet port and a fluid outlet port and is configured such that a H/C fluid may pass through the plurality of hollow fibers.

Example 12 is the device of Example 6, wherein the remote monitoring unit includes a display adapted to display the inlet blood pressure, the outlet blood pressure and a pressure drop based on a difference between the inlet and outlet blood pressures.

Example 13 is the device of Example 14, wherein the heat exchanger module includes an H/C fluid inlet chamber and an H/C fluid outlet chamber, and the oxygenator module includes a gas inlet port and a gas outlet port.

Example 14 is an oxygenation system for use in connection with extracorporeal blood circulation, the system comprising: an oxygenator housing including a blood inlet end cap defining a blood inlet opening, a blood outlet end cap defining a blood outlet and a blood flow path between the blood inlet and the blood outlet, the oxygenator housing defining a heat exchanger module and an oxygenator module adjacent the heat exchanger module; a plurality of stacked, mat layers of hollow fibers disposed inside the housing and along the blood flow path, the hollow fibers fluidly coupled to the oxygenator module; a blood inlet port having a blood inlet lumen defining an inlet sensing hole and fluidly coupled to the blood inlet opening of the blood inlet end cap, the blood inlet port including an inlet pressure sensor housing in fluid communication with the blood inlet lumen through the inlet sensing hole, the inlet pressure sensor housing adapted to house an inlet pressure sensor, wherein the inlet sensing hole is disposed less than 3 cm from the blood inlet opening on the blood inlet end cap; a blood outlet port having a blood outlet lumen fluidly defining an outlet sensing hole and fluidly coupled to the blood outlet opening of the blood outlet end cap, the blood outlet port including an outlet pressure sensor housing in fluid communication with the blood outlet lumen through the outlet sensing hole, the outlet pressure sensor housing adapted to house an outlet pressure sensor; and a remote monitoring unit communicatively coupled to the inlet pressure sensor and outlet pressure sensor, wherein during operation of the oxygenation system, the inlet pressure sensor is configured to generate an inlet blood pressure signal and the outlet pressure sensor is configured to generate an inlet blood pressure signal, and wherein the monitoring unit is configured to receive the inlet blood pressure signal and outlet blood pressure signal and display corresponding blood pressure measurements.

Example 15 is the system of Example 14, wherein the heat exchanger module includes an H/C fluid inlet chamber and an H/C fluid outlet chamber, and the oxygenator module includes a gas inlet port and a gas outlet port.

Example 16 is the system of Example 15, wherein the H/C fluid may pass from the H/C fluid inlet chamber through the plurality of hollow fibers and exit to the H/C outlet chamber.

Example 17 is the system of Example 15, wherein a gas mixture may pass from the gas inlet port through the plurality of fibers and exit through the gas outlet port.

Example 18 is the system of Example 14, wherein the remote monitoring unit is adapted to communicate with the remote monitoring unit by wireless telemetry.

Example 19 is the system of Example 14, wherein the remote monitoring unit is adapted to communicate with the remote monitoring unit via electrical connectors mechanically coupled between the inlet and outlet pressure sensors and the remote monitoring unit.

Example 20 is the system of Example 14, wherein the remote monitoring unit includes a display and an alarm.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic view of a patient undergoing extracorporeal blood circulation.

FIG. 2 is a schematic front view of an exemplary stacked oxygenator device, which does not include pressure sensors.

FIG. 3 is a lateral isometric view from the left of the oxygenator device of FIG. 2.

FIG. 4 is a lateral cross-section of the oxygenator device taken along line A-A of FIG. 2.

FIG. 5 is an exploded view of the oxygenator device of FIG. 2.

FIG. 6 is an exploded view of a disposable pressure sensor adapted to be integrated into the blood inlet/outlet ports of FIGS. 2 to 5 oxygenator.

FIG. 7 is a lateral cross-section of FIGS. 2 to 5 oxygenator with pressure sensors of FIG. 6 integrated into the blood inlet/outlet ports, taken along the line A-A of FIG. 2.

FIG. 8 is a perspective view of the oxygenator shown in FIG. 7.

FIG. 9 is a lateral cross-section of a second embodiment of the oxygenator of FIGS. 2 to 5 with pressure sensors of FIG. 6 integrated into the blood inlet/outlet ports, taken along line B-B of FIG. 2.

FIG. 10 is a perspective view of the oxygenator of FIG. 9.

FIG. 11 is a perspective view of the oxygenator of FIG. 10, showing a third embodiment of the invention.

While the invention is amenable to various modifications and alternative forms, specific embodiments have been shown by way of example in the drawings and are described in detail below. The intention, however, is not to limit the invention to the embodiments described. On the contrary, the invention is intended to cover all modifications, equivalents, and alternatives falling within the scope of the invention as defined by the appended claims.

### DETAILED DESCRIPTION

The following detailed description is exemplary in nature and is not intended to limit the scope, applicability, or configuration of the invention in any way. Rather, the following description provides some practical illustrations for implementing exemplary embodiments of the present invention. Examples of constructions, materials, and/or dimensions are provided for selected elements. Those skilled in the art will recognize that many of the noted examples have a variety of suitable alternatives.

As the terms are used herein with respect to measurements (e.g., dimensions, characteristics, attributes, components, etc.), and ranges thereof, of tangible things (e.g., products, inventory, etc.) and/or intangible things (e.g., data, electronic representations of currency, accounts, information, portions of things (e.g., percentages, fractions), calculations, data models, dynamic system models, algorithms, parameters, etc.), “about” and “approximately” may be used, interchangeably, to refer to a measurement that includes the stated measurement and that also includes any measurements that are reasonably close to the stated measurement, but that may differ by a reasonably small amount such as will be understood, and readily ascertained, by individuals having ordinary skill in the relevant arts to be attributable to measurement error; differences in measurement and/or manufacturing equipment calibration; human error in reading and/or setting measurements; adjustments made to optimize performance and/or structural parameters in view of other measurements (e.g., measurements associated with other things); particular implementation scenarios; imprecise adjustment and/or manipulation of things, settings, and/or measurements by a person, a computing device, and/or a machine; system tolerances; control loops; machine-learning; foreseeable variations (e.g., statistically insignificant variations, chaotic variations, system and/or model instabilities, etc.); preferences; and/or the like.

Although illustrative methods may be represented by one or more drawings (e.g., flow diagrams, communication flows, etc.), the drawings should not be interpreted as implying any requirement of, or specific order among or between, various steps disclosed herein. However, certain some embodiments may require certain steps and/or certain orders between certain steps, as may be explicitly described herein and/or as may be understood from the nature of the steps themselves (e.g., the performance of some steps may depend on the outcome of a previous step). Additionally, a “set,” “subset,” or “group” of items (e.g., inputs, algorithms, data values, etc.) may include one or more items, and, similarly, a subset or subgroup of items may include one or more items. A “plurality” means more than one.

As used herein, the term “based on” is not meant to be restrictive, but rather indicates that a determination, identification, prediction, calculation, and/or the like, is performed by using, at least, the term following “based on” as an input. For example, predicting an outcome based on a particular piece of information may additionally, or alternatively, base the same determination on another piece of information.

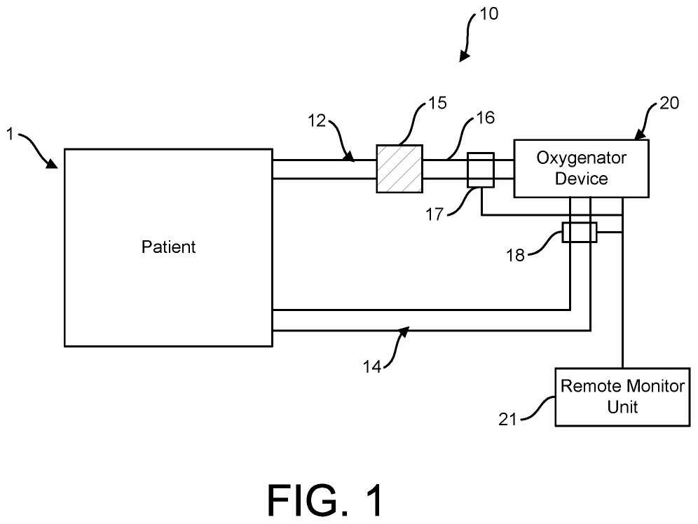

FIG. 1 is a schematic view of an extracorporeal blood circulation system 10 (also referred to herein as an extracorporeal circuit 10) for supporting a patient 1 requiring extracorporeal blood circulation. In various embodiments, the patient 1 is connected through a first tubing 12 (also called a venous line) to the extracorporeal blood circuit 10 including a pump 15 to cause blood to be transferred from the patient 1, through the first tubing 12 and 16, to a mass transfer device 20, commonly referred to as an oxygenator device 20 (or oxygenator 20). Note that the oxygenator 20 may include one or both of an oxygenator module and a heat exchanger module.

The system 10 further includes a second tubing 14 (also called an arterial line) that extends from the oxygenator 20 to the patient 1 for transferring blood that has been circulated within the pump 15 and oxygenator 20 back to the patient 1. The extracorporeal circuit 10 includes a plurality of sensors which measure parameters like blood pressures, flow rate, temperatures, hematocrit, oxygen saturation and oxygen and carbon dioxide partial pressures of blood, that must be kept under control during the perfusion process. In general, and not exclusively, such sensors may be located pre- and/or post-oxygenator, depending on whether the quantities must be measured on the venous or the arterial side. In FIG. 1, a group of venous side sensors 17 and a group of arterial side sensors 18 are schematically shown. They may be in direct contact with blood or may measure the quantities from the tubing outside and are electrically connected to a separate and remote control and monitoring unit 21 under operator (e.g., a perfusionist) control by means of an appropriate control system. In various embodiments, oxygen (O₂) and carbon dioxide (CO₂) are exchanged between blood and a gas mixture within the oxygenator device 20, as will be described further herein. The oxygenator device 20, in certain embodiments, is also configured for exchanging temperature (hot or cool temperatures) between the blood and heating/cooling (H/C) fluid within the oxygenator device 20. In some embodiments, the H/C fluid is water or a water solution.

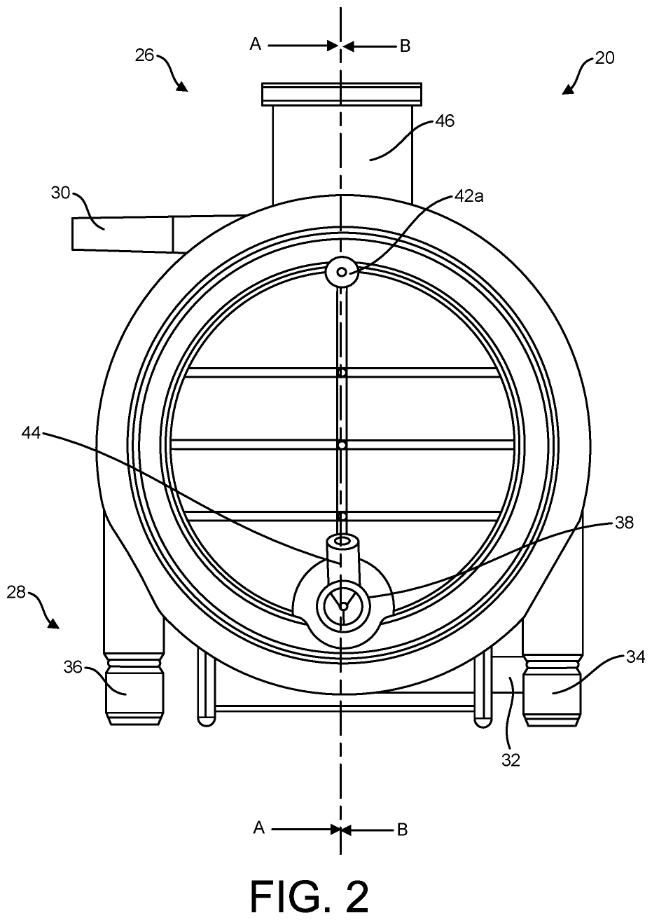

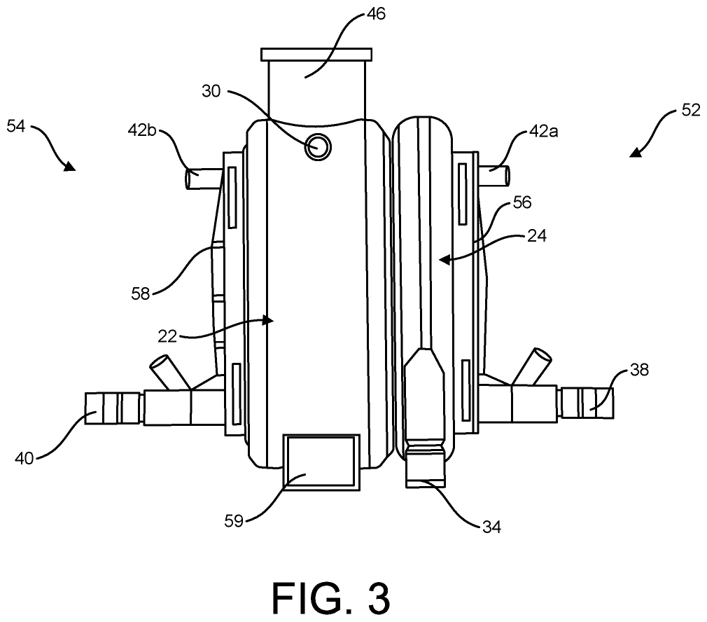

FIG. 2 is a front view of an exemplary stacked oxygenator device 20 having an oxygenator module (not shown) and a heat exchanger module (not shown). The oxygenator device 20 has an upper portion 26 and a lower portion 28. In various embodiments, the oxygenator 20 includes a gas inlet port 30 configured for receiving a gas mixture, a gas outlet port 32 configured for exporting a gas mixture, a H/C fluid inlet port 34 for receiving H/C fluid, an H/C fluid outlet port 36 for exporting H/C fluid, a blood inlet port 38 for receiving blood from the patient 1 through the tubing 16, and a blood outlet port (not shown) for exporting blood from the oxygenator device 20 back to the patient 1 through the tubing 14. The oxygenator device 20 further includes a venous sampling port 44 and a first purging port 42a, as will be described further with reference to FIG. 3. In some embodiments, the oxygenator device 20 additionally includes a bracket attachment 46 to allow for attachment and rotation of the oxygenator device 20 from the top. Alternatively, the bracket attachment 46 may also be coupled to the bottom so as to allow attachment and rotation of the oxygenator from the bottom.

FIG. 3 is a side view, from the left of FIG. 2, of the oxygenator device 20. As illustrated, the oxygenator device 20 includes a front portion 52 and a rear portion 54. The front portion 52 includes a blood inlet end cap 56 coupled to a blood inlet port 38 configured for receiving blood from tubing 16 and the rear portion 54 includes a blood outlet end cap 58 coupled to a blood outlet port 40 for providing an exit for the blood to return to the patient 1 through the tubing 14. Additionally, as illustrated in FIG. 3, the oxygenator device 20 includes the gas inlet port 30 on the side surface of the oxygenator device 20. The oxygenator device 20 additionally includes a plurality of purging ports 42 including at least the first purging port 42a and a second purging port 42b. Purging ports 42a, 42b may allow for removal of air during an initial priming phase of the oxygenator device 20 prior to use with the patient. During operation, i.e., when blood and gas flow through the device 20, the purging ports 42a, 42b may be reopened for removing entrapped air from blood. Additionally, the purging ports 42a, 42b may be opening after operation of the device 20, i.e., when blood is no longer flowing through the device 20, to ensure proper emptying of any blood from the device 20 and returning it to the patient. The oxygenator device 20 may also include a pedestal 59 positioned on a bottom surface of the device 20 for supporting and stabilizing the device 20, if the bracket attachment 46, as shown in FIG. 2, is located at the top of the oxygenator.

As previously described with reference to FIG. 2, the oxygenator device 20 includes a front portion 52 and a rear portion 54. In these embodiments and as illustrated in FIG. 3, the front portion 52 encompasses at least a portion of the heat exchanger module 24 and the rear portion 54 encompasses at least a portion of the oxygenator module 22.

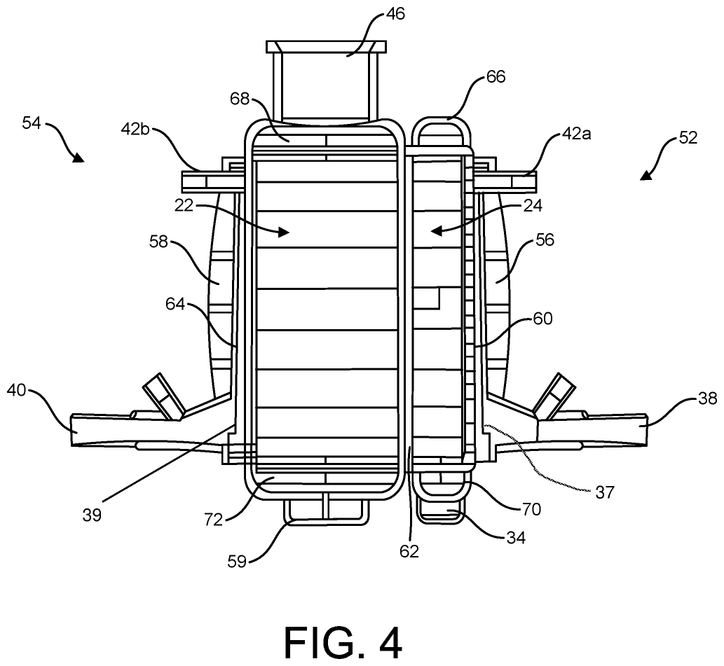

FIG. 4 illustrates a lateral cross-section of the oxygenator device 20 taken along line A-A of FIG. 2. In the illustrative embodiment of FIG. 4, the oxygenator device 20 includes the oxygenator module 22 and the heat exchanger module 24 positioned adjacent the oxygenator module 22. Both modules 22, 24 are provided with hollow fiber mat layers vertically stacked one adjacent to the other. As shown, the inlet end cap 56 includes an inlet opening 37 coupled to the inlet blood port 38 and the outlet end cap 58 includes an outlet opening 39 coupled to the outlet blood port 40.

The heat exchanger module 24 is bordered on a right side, towards the front portion 52 of the oxygenator device 20, by a blood inlet distribution grid 60. The blood inlet distribution grid 60 receives the inputted blood from the blood inlet port 38 connected to the inlet opening 37 of the inlet end cap 56 and distributes it within the blood inlet distribution grid 60 before flowing the blood into the heat exchanger module 24. Heat exchanger module 24 is bordered on the left side, towards the rear portion 54, by a separation grid 62 which provides a physical separation between the oxygenator module 22 and the heat exchanger module 24 and distributes blood flowing past the heat exchanger module 24 towards the oxygenator module 22. The heat exchanger module 24 is thus on the opposing side of separation grid 62 relative to the oxygenator module 22 which is positioned further towards the rear portion 54 of the oxygenator device 20. The heat exchanger module 24 is positioned vertically below an H/C fluid inlet chamber 66 and positioned vertically above the pedestal 59 and an H/C fluid outlet chamber 70. The oxygenator module 22 is also bordered on the left side, towards rear portion 54 of the oxygenator device 20, by a blood outlet collection grid 64 which is configured for collecting the blood flowing from the oxygenator module 22 and directing it through the outlet opening 39 of the outlet end cap 58 to the blood outlet port 40. As illustrated, vertically positioned above the oxygenator module 22 is a gas inlet chamber 68 and the bracket attachment 46. Vertically positioned below the oxygenator module 22 is the gas outlet chamber 72.

Each of the oxygenator module 22 and the heat exchanger module 24 generally include two portions, or two halves. As will be further described below, the oxygenator module 22 has a portion configured for communication with the gas inlet chamber 68 and a portion configured for communication with the gas outlet chamber 72. Similarly, the heat exchanger module 24 has a portion configured for communication with the H/C fluid inlet chamber 66 and a portion configured for communication with the H/C fluid outlet chamber 70. As illustrated, the front portion 52 of the oxygenator device 20 includes the blood inlet port 38 and the rear portion 54 of the oxygenator device 20 includes the blood outlet port 40. As a result of this configuration, when the blood is driven to pass through both the heat exchanger module 24 and the oxygenator module 22 it flows along a blood flow path from the blood inlet port 38 to the blood outlet port 40, the blood is able to come into contact (by interposition of the appropriate hollow fiber membranes in the heat exchanger module 24 and the oxygenator module 22), with the fluid mixtures and the gas for sufficient heat and gas exchange.

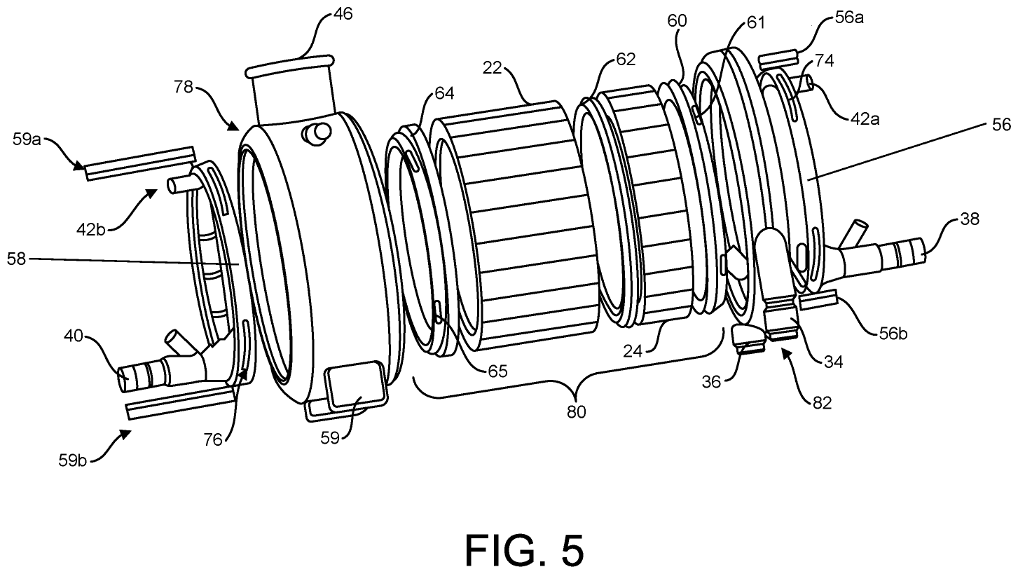

FIG. 5 is an exploded view of the oxygenator device 20 illustrating the component assembly within the oxygenator device 20. As shown, the oxygenator device 20 includes a blood path comprising the blood inlet end cap 56 (with the blood inlet port 38), the purging port 42a, the potted body 80, and the blood outlet end cap 58 (with the blood outlet port 40) and the purging port 42b. In various embodiments, the inlet port 38 is a separate component from the inlet end cap 56 and may be coupled or assembled with it by resin casting, such that the internal lumen of the inlet opening 37 is continuous with the internal lumen of the inlet port 38. Similarly, in various embodiment, the blood outlet port 40 is a separate component from the outlet end cap 58 and may be coupled or assembled with it by resin casting, such that the internal lumen of the outlet opening 39 is continuous with the internal lumen of the outlet port 40. The potted body 80 includes, embedded all together in one (potted) piece, the blood inlet distribution grid 60, the heat exchanger module 24, the separation grid 62, the oxygenator module 22, and the blood outlet collection grid 64. Access for H/C fluid and gas to the inner lumens of the hollow fibers of the heat exchanger 24 and oxygenator 22 is made possible through the hollow fiber open ends on the potted body outer surface.

In certain embodiments, the blood inlet end cap 56 is provided with a plurality of peripheral cavities 74 that mechanically fit into the corresponding peripheral notches 61 on the blood inlet distribution grid 60 of the potted body 80. Air tightness between blood inlet end cap 56 and blood inlet distribution grid 60 may be obtained by resin casting along the two circular contact surfaces of the blood inlet end cap 56 and the blood inlet distribution grid 60. Similarly, in various embodiments, on the opposite end of the potted body 80, the blood outlet end cap 58 includes a plurality of peripheral cavities 76 that mechanically fit into the corresponding peripheral notches 65 on the outlet collection grid 64 of the potted body 80. Air tightness between blood outlet end cap 58 and outlet collection grid 64 is obtained by resin casting along the circular contact surfaces of the blood outlet end cap 58 and outlet collection grid 64. In this way, the entire blood compartment, including blood inlet end cap 56, blood outlet end cap 58, potted body 80 and blood inlet/outlet ports 38 and 40 are joined in one airtight piece.

During operation, blood enters the oxygenator device 20 through the blood inlet port 38 in a direction orthogonal to the stacked hollow fiber mat layers and continues into the device through the blood inlet opening 37 of the blood inlet end cap 56, then crosses blood inlet distribution grid 60, the stacked hollow fiber mat layers forming the heat exchanger module 24, the separation grid 62, the stacked hollow fiber mat layers forming the oxygenator 22, the outlet collection grid 64, and exits the device in a direction orthogonal to the stacked hollow fiber layers by flowing through the outlet opening 39 of the blood outlet end cap 58 and through the blood outlet port 40. As shown in FIGS. 3 to 5, the blood inlet and blood outlet paths (e.g., the ports 38 and 40) to reach the inside of the oxygenator 20 are quite short. In certain embodiments, the blood inlet distribution grid 60, the separation grid 62, and the outlet collection grid 64 are circular plastic parts with relatively large bores (from 1 to 8 mm) throughout their surfaces and are configured for keeping the elements of the heat exchanger module 24 and the oxygenator module 22 in place and assuring an even distribution of blood flowing across them. The remaining parts of the device 20 comprise external housings enclosing the H/C fluid compartment and the gas compartment, i.e., the H/C fluid collector 82 (including the H/C fluid inlet port 34 and the H/C fluid outlet port 36) and the gas collector 78 (including the gas inlet port 30 and the gas outlet port 32, as shown in FIG. 2, and including the bracket attachment 46 and the pedestal 59).

As shown in FIG. 5, the H/C fluid collector 82 is positioned externally over the potted body 80 portion corresponding to the heat exchanger module 24 and assembled to the blood inlet end cap 56 by means of resin casting to air tighten the circular right side of the H/C fluid compartment. The left circular edge of the H/C fluid collector 82 is positioned externally in correspondence of the separation grid 62 and is air tightened to the potting body 80 by resin casting. In this way, the entire H/C fluid compartment is air tightened. The H/C fluid compartment is divided into two halves including an H/C fluid inlet chamber 66 and an H/C fluid outlet chamber 70 (shown in FIG. 4) by means of two sealing gaskets 56a, 56b which are inserted along two alignment features, illustratively two diametrically opposed and longitudinal grooves located on the outer surface of the potted body 80. The H/C fluid flows inside the H/C fluid compartment to (and from) the heat exchanger module 24 through the gap between the inner surface of the H/C fluid collector 82 and the outer surface of the potted body 80, thus forming the H/C fluid inlet chamber 66 and the H/C fluid outlet chamber 70 (shown in FIG. 4).

Similarly, as shown, the gas collector 78 is positioned externally in correspondence with the outer surface of the potted body 80 relative to the oxygenator module 22 and assembled with the blood outlet end cap 58 by means of resin casting in order to air tighten the left circular side of the gas compartment of the device 20. The right circular edge of the gas collector 78 is positioned externally next to the H/C fluid collector 82 in correspondence of the separation grid 62 and is air tightened to the potting body 80 by resin casting. In this way, the gas compartment is entirely air tightened. Also, the gas compartment is divided in two halves: a gas inlet chamber 68 and a gas outlet chamber 72 (shown in FIG. 4) by means of two sealing gaskets 59a, 59b which are inserted along two alignment features, illustratively two diametrically opposed and longitudinal grooves located on the outer surface of the potted body 80. The gas flows to (and from) the oxygenator module 22 through the gas compartment given by the gap between the inner surface of gas collector 78 and outer surface of the potted body 80, forming the gas inlet chamber 68 and the gas outlet chamber 72.

In some embodiments, the body 80 of the oxygenator device 20 is obtained by stacking circular layers of hollow fiber mats, made, for the oxygenator, of polypropylene, or polymethilpentene and, for the heat exchanger, of polyethylene, or polyurethane, potting it with polyurethane resin and afterwards slicing its outer surface to cut open the fibers lumens so as to allow water and gas circulation inside the fiber lumens. The woven fibers of each layer are alternatively angled vs an alignment direction by an angle α and an angle β disposed on opposite sides of the alignment direction. Angles α and β may be equal, or not, and are each comprised in the range 0 to 25 degrees. To give them a certain structural consistency, the layers are individually and circularly hot sealed on their external circumference. During such an operation, two orienting elements, whose function is to ease the correct stacking of the layers during the subsequent assembly of the body 80 prior to potting, are also hot sealed along the outer circumference of each layer.

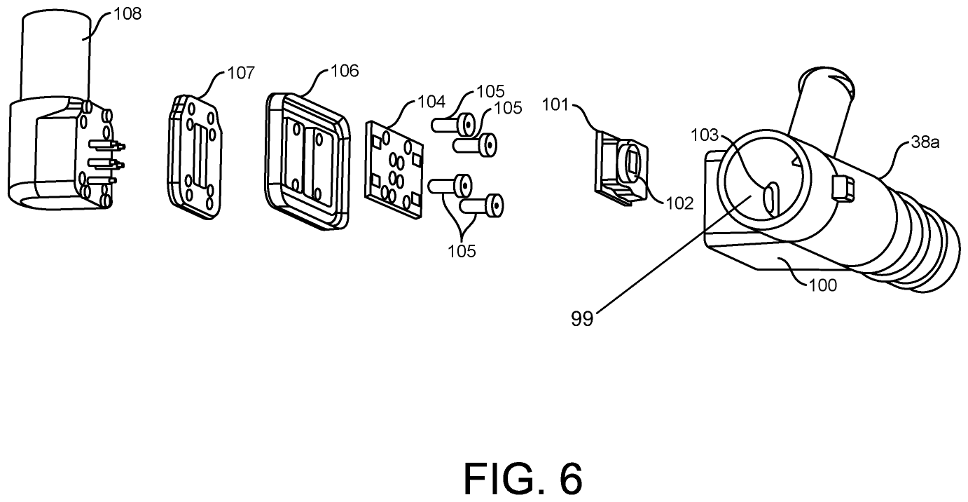

FIG. 6 shows an exploded view of the blood inlet port 38 (or outlet port 40) adapted to accommodate pressure sensor integration. In various embodiment, the blood port 38 or 40 is separate from the blood end cap (either 56 or 58 shown in FIG. 5) and assembled to it by resin casting. Note that while further details are provided below with respect to the blood inlet port 38, the same details also apply in certain embodiments to the blood outlet port 40.

The blood inlet port 38 is fluidly coupled to the blood inlet opening of the blood inlet end cap 56 via an inlet port 38a. As shown, the inlet port 38a includes a blood inlet lumen 99. The inlet port 38a includes an inlet sensing hole 103 in fluid communication with the blood inlet lumen 99. The inlet port 38 also include an inlet pressure sensor housing 100. The inlet pressure sensor housing 100 is adapted to accept and support a pressure sensor 101, such as inlet pressure sensor 101, which is introduced with the circular sensing element 102 facing, through the inlet sensing hole 103, the blood flow and being in direct contact with it. The pressure sensor 101 is in fluid communication with the blood inlet lumen 99 through the inlet sensing hole 103. The pressure sensor 101 can be any type of commercially available devices, like, for instance, Amphenol Nova Sensors NPC 100 or 120 series. In addition to the sensing element 102, the pressure sensor 101 further includes circuitry for converting the pressure sensed by the sensing element 102 to an appropriate electrical signal.

While FIG. 6 is shown with respect to an inlet blood port 38, it could be similarly illustrated with respect to the outlet blood port 40. For instance, the blood outlet port 40 is fluidly coupled to the blood outlet opening of the blood outlet end cap 58 via an outlet port, which is configured similarly to inlet port 38a. The blood outlet port includes a blood outlet lumen, configured similarly to lumen 99. The outlet port includes an outlet sensing hole, which is configured similarly to inlet sensing hole 103, in fluid communication with the blood inlet lumen. The outlet port 40 also includes an outlet pressure sensor housing, configured similarly to inlet pressure sensor housing 100. The outlet pressure sensor housing is adapted to accept and support a pressure sensor 101, such as an outlet pressure sensor, which is introduced with the circular sensing element 102 facing, through the outlet sensing hole, the blood flow and being in direct contact with it. The outlet pressure sensor is in fluid communication with the blood outlet lumen through the outlet sensing hole.

As shown, the inlet blood port 38a is configured such that the sensing hole 103 is positioned very proximate to the blood inlet opening 37 on the blood inlet end cap 56. This proximity is enabled by the structure of the stacked oxygenator where the inlet blood port can be positioned close to the blood entrance into the gas exchange compartment of the oxygenator. In various embodiments, the sensing hole 103 is located at a distance of less than 3 cm from the inlet opening 37. In other embodiments, the sensing hole 103 is located at a distance of less than 2 cm from the inlet opening 37. In some embodiments, the sensing hole 103 is located 1.5 cm from the inlet opening 37. This minimal distance between the sensing hole 103 and the inlet opening 37, substantially reduces the pressure drop or loss associated with the flow through the inlet blood port between the inlet opening the pressure sensor. This, in turn, allows the pressure sensor to measure a more accurate reading of the pressure at the inlet (or outlet) to the end caps, thereby allowing for a more accurate determination of the pressure drop across the device.

In certain embodiments, the pressure sensor 101, including the sensing element 102, is assembled and sealed to the blood port by resin casting to prevent blood leaks and is electrically connected by means of wires (not shown) to the board 104, which includes terminals for electrically coupling to a cable or wireless transmission system. The board 104 is assembled to the covers 106 and 107 using the four screws 105. The outside cover 106, which houses the board 104 and screws 105 is glued and sealed to the blood port where the pressure sensor 101 has already been encased. The pins of the electrical connector 108 can be introduced into corresponding openings in the covers 106 and 107 and can contact the electrical terminals of the board 104 to which the wires to the pressure sensor 101 are connected. While the electrical connector 108 is shown in a particular orientation in FIG. 6, in other embodiments, the electrical connector 108 may be oriented in any direction with respect to the blood ports 38a, 40a.

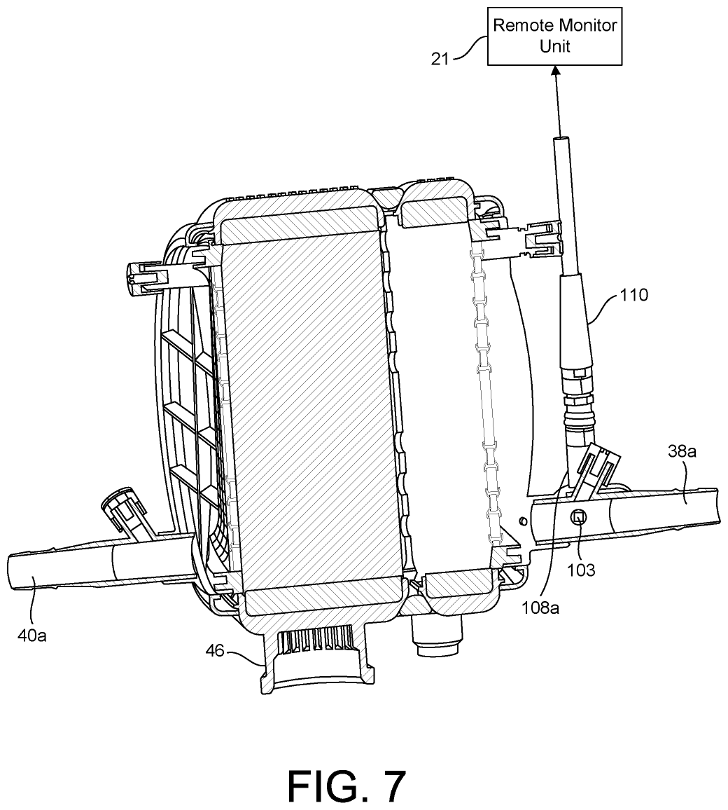

FIG. 7 is a cross-section, along the line A-A in FIG. 2, of a first embodiment of this application showing a stacked oxygenator of the type described in FIGS. 2 to 5 with the pressure sensors integrated in the blood inlet and outlet ports and connected by means of two separate cables to the remote control and monitoring unit 21. While the connectors and cables are shown in a particular orientation in FIG. 7, these components may be oriented in any other direction. One of the two cables, the inlet pressure cable 110 is shown in FIG. 7. An end of the cable 110 is inserted into the connector 108a of the integrated inlet port pressure sensor. Also, the oxygenator is shown with the bracket attachment 46 located at the bottom (as opposed to the top position shown in FIGS. 2 to 5). In FIG. 7 the pressure sensing hole 103 (for the blood inlet port 38a) is visible. For the blood outlet port 40a, the integrated pressure sensor is not visible.

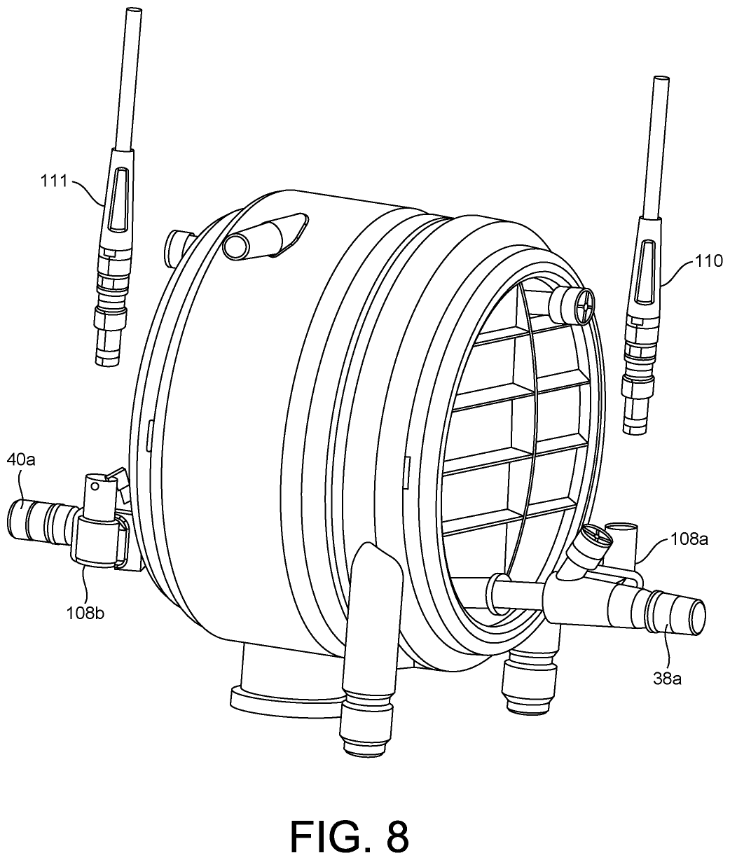

FIG. 8 is a perspective view of the oxygenator shown in FIG. 7. The two electrical cables 110 (for the inlet pressure sensor) and 111 (for the outlet pressure sensor) are disconnected from the two pressure sensors 108a and 108b, respectively integrated into the inlet port 38a and outlet port 40a. They transfer the pressure signals to the remote control and monitoring unit 21.

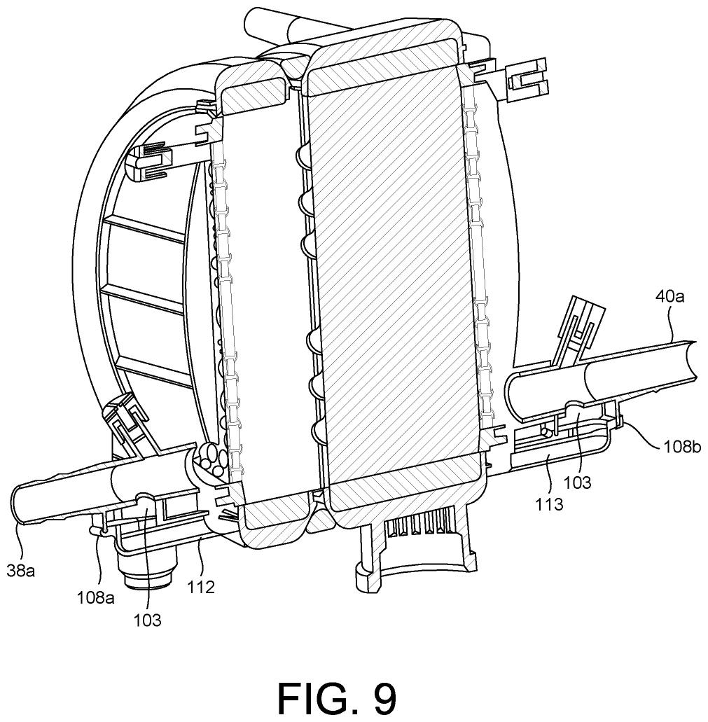

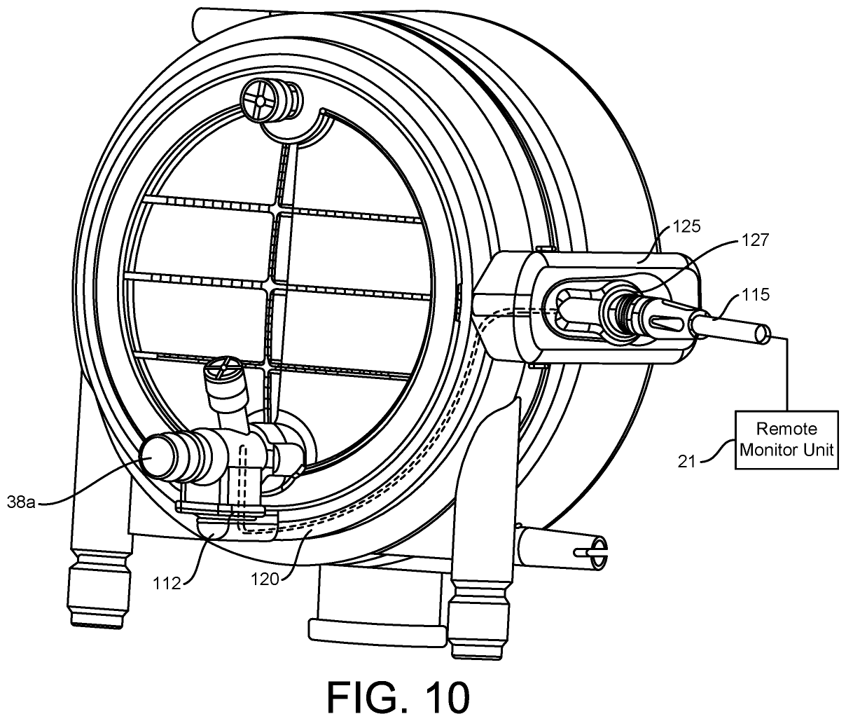

FIGS. 9 and 10 are respectively a cross-sectional view along line B-B in FIG. 2, and a perspective view of a second embodiment of this application showing the oxygenator described in FIGS. 2 to 5 with the pressure sensors integrated in the blood inlet and outlet ports and connected by means of a single cable to the remote monitoring unit. Such an embodiment provides further simplification and compactness to the whole oxygenator device compared to the one presented in FIGS. 7 and 8. The two blood ports 38a and 40a integrate the two pressure sensors 108a and 108b now oriented to present the sensing holes 103 in the lower half of the two port internal lumens. The sensor wires travel following either a hidden dotted path inside the channel 112 (and corresponding 113 on the outlet port) and 120 (and correspondingly on the outlet side of the oxygenator, not visible in FIG. 10) or a visible printed circuit board (e.g., a Kapton PCB) following the same path to reach the unique female connector pad 125 conveniently placed in a central position on one side of the oxygenator housing. Pad 125 has a terminal connector 127, which may be oriented in any direction including and in addition to the orientation shown in FIGS. 9 and 10, where the cable can be attached to bring the pressure signals to the remote control and monitoring unit 21.

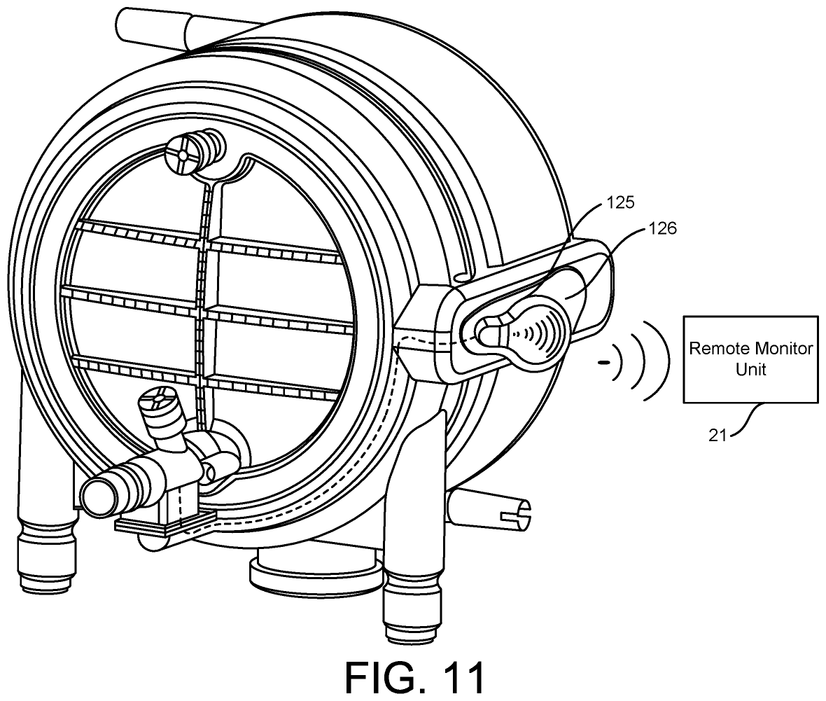

FIG. 11 shows a perspective view of the oxygenator of FIGS. 9 and 10 with a third embodiment including a wireless (e.g., radio-frequency, Bluetooth or Wi-Fi) connection between the sensors and the remote control and monitoring unit. In this case, the pad 125 besides the wiring to the integrated pressure sensors (as already described for FIGS. 9 and 10), includes the battery power supply and the telemetry transmitting circuitry 126 to make it possible the wireless connection to the remote control and monitoring unit 21. This embodiment provides further simplification to the sensor/oxygenator integration.

Once the pressure signals from the sensors reach the remote control and monitoring unit they may be displayed in mmHg (which is the most commonly used unit in cardiac surgery or ECMO applications), shown on a screen in numerical, or graphical form and compared with limits set by the operator, or fixed by the system. If such limits are exceeded, audible and visual alarms may be switched on to attract the operator attention who may decide to start corrective actions. Inlet/outlet pressure values may also be shown as difference between them (i.e., as a pressure drop, DeltaP). The pressure drop value may be monitored by activating an alarm, in case it exceeds a set value. It may also be factored into an appropriate functionality of the remote control and monitoring unit to automatically act on the pump speed (i.e., revolutions per minute) so as to maintain the blood flow rate at a constant set value.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. An oxygenation device for use in connection with extracorporeal blood circulation, the device comprising:
an oxygenator housing including a blood inlet end cap defining a blood inlet opening, a blood outlet end cap defining a blood outlet and a blood flow path between the blood inlet and the blood outlet;
a plurality of stacked, mat layers of hollow fibers disposed inside the housing and along the blood flow path, the hollow fibers fluidly coupled to a gas inlet port and a gas outlet port;
a blood inlet port having a blood inlet lumen defining an inlet sensing hole and fluidly coupled to the blood inlet opening of the blood inlet end cap, the blood inlet port including an inlet pressure sensor housing in fluid communication with the blood inlet lumen through the inlet sensing hole, the inlet pressure sensor housing adapted to house an inlet pressure sensor, wherein the inlet sensing hole is disposed less than 3 cm from the blood inlet opening on the blood inlet end cap; and
a blood outlet port having a blood outlet lumen fluidly defining an outlet sensing hole and fluidly coupled to the blood outlet opening of the blood outlet end cap, the blood outlet port including an outlet pressure sensor housing in fluid communication with the blood outlet lumen through the outlet sensing hole, the outlet pressure sensor housing adapted to house an outlet pressure sensor;
wherein during operation of the oxygenation device, each of the inlet pressure sensor and the outlet pressure sensor are configured to measure an inlet blood pressure and an outlet blood pressure, respectively.

2. The device of claim 1, wherein the oxygenator housing defines an oxygenator module and a heat exchanger module, and further wherein the plurality of hollow fibers includes oxygenator fibers associated with the oxygenator module and heat exchanger fibers associated with the heat exchanger module.

3. The device of claim 2, wherein the housing further comprises a separation grid or a collection grid positioned between the heat exchanger module and the oxygenator module.

4. The device of claim 1 further comprising a remote monitoring unit communicatively coupled to each of the inlet and outlet pressure sensors.

5. The device of claim 4 further comprising communication circuitry coupled to each of the inlet and outlet pressure sensors and adapted to communicate with the remote monitoring unit.

6. The device of claim 5, wherein the communication circuitry is adapted to communicate with the remote monitoring unit by wireless telemetry.

7. The device of claim 1 further comprising electrical terminals mechanically connected to each of the inlet and outlet ports and adapted for coupling at least one of the inlet and outlet pressure sensors with an electrical connector.

8. The device of claim 7, wherein the electrical connector is configured to communicatively couple both of the pressure sensors with a remote monitoring unit.

9. The device of claim 7 further comprising a second electrical connector and wherein the first electrical connector is adapted to couple the inlet pressure sensor to the remote monitoring unit and the second electrical connector is adapted to couple the outlet pressure sensor to the remote monitoring unit.

10. The device of claim 1, wherein the housing is configured such that a gas mixture may enter through the gas inlet port, pass through the plurality of hollow fibers and exit through the gas outlet port.

11. The device of claim 1, wherein the housing further defines a fluid inlet port and a fluid outlet port and is configured such that a H/C fluid may pass through the plurality of hollow fibers.

12. The device of claim 6, wherein the remote monitoring unit includes a display adapted to display the inlet blood pressure, the outlet blood pressure and a pressure drop based on a difference between the inlet and outlet blood pressures.

13. The device of claim 6, wherein the remote monitoring unit includes an alarm configured to alert a user upon any of the inlet blood pressure, the outlet blood pressure and a difference between the inlet and outlet blood pressure achieving a certain set value.

14. An oxygenation system for use in connection with extracorporeal blood circulation, the system comprising:
an oxygenator housing including a blood inlet end cap defining a blood inlet opening, a blood outlet end cap defining a blood outlet and a blood flow path between the blood inlet and the blood outlet, the oxygenator housing defining a heat exchanger module and an oxygenator module adjacent the heat exchanger module;
a plurality of stacked, mat layers of hollow fibers disposed inside the housing and along the blood flow path, the hollow fibers fluidly coupled to the oxygenator module;
a blood inlet port having a blood inlet lumen defining an inlet sensing hole and fluidly coupled to the blood inlet opening of the blood inlet end cap, the blood inlet port including an inlet pressure sensor housing in fluid communication with the blood inlet lumen through the inlet sensing hole, the inlet pressure sensor housing adapted to house an inlet pressure sensor, wherein the inlet sensing hole is disposed less than 3 cm from the blood inlet opening on the blood inlet end cap;
a blood outlet port having a blood outlet lumen fluidly defining an outlet sensing hole and fluidly coupled to the blood outlet opening of the blood outlet end cap, the blood outlet port including an outlet pressure sensor housing in fluid communication with the blood outlet lumen through the outlet sensing hole, the outlet pressure sensor housing adapted to house an outlet pressure sensor; and
a remote monitoring unit communicatively coupled to the inlet pressure sensor and outlet pressure sensor, wherein during operation of the oxygenation system, the inlet pressure sensor is configured to generate an inlet blood pressure signal and the outlet pressure sensor is configured to generate an inlet blood pressure signal, and wherein the monitoring unit is configured to receive the inlet blood pressure signal and outlet blood pressure signal and display corresponding blood pressure measurements.

15. The system of claim 14, wherein the heat exchanger module includes an H/C fluid inlet chamber and an H/C fluid outlet chamber, and the oxygenator module includes a gas inlet port and a gas outlet port.

16. The system of claim 15, wherein the H/C fluid may pass from the H/C fluid inlet chamber through the plurality of hollow fibers and exit to the H/C outlet chamber.

17. The system of claim 15, wherein a gas mixture may pass from the gas inlet port through the plurality of fibers and exit through the gas outlet port.

18. The system of claim 14, wherein the remote monitoring unit is adapted to communicate with the remote monitoring unit by wireless telemetry.

19. The system of claim 14, wherein the remote monitoring unit is adapted to communicate with the remote monitoring unit via electrical connectors mechanically coupled between the inlet and outlet pressure sensors and the remote monitoring unit.

20. The system of claim 14, wherein the remote monitoring unit includes a display and an alarm.
